(19) United States
(12) Patent Application Publication
(10) Pub. No.: US 2025/0032742 A1
(43) Pub. Date: Jan. 30, 2025

(54) SYSTEMS AND METHODS FOR ATTACHMENTS TO DEVICES FOR NORMALIZING BREATHING THROUGH PRESSURIZED OXYGEN

(71) Applicant: Bowman Patents, LLC, Huntsville, AL (US)

(72) Inventor: Jerry Bowman, Huntsville, AL (US)

(73) Assignee: Bowman Patents, LLC, Huntsville, AL (US)

(21) Appl. No.: 18/225,882

(22) Filed: Jul. 25, 2023

(51) Int. Cl. A61M 16/08

(52) U.S. Cl. CPC A61M 16/0816; A61M 16/0875; A61M 2202/0208

(57) ABSTRACT

A device of the present disclosure has an attachment section configured for attaching to a pressurized oxygen delivery system and covering vent holes on the pressurized oxygen delivery system configured for venting exhaled carbon dioxide (CO²). The device also has a tube fixedly coupled to the attachment section on a first end of the tube and a plurality of vent holes on a second end of the tube configured for eliminating the CO².

### BACKGROUND

There exist different devices that aid an individual in normalized breathing through pressurized oxygen. For example, a continuous positive airway pressure (C-PAP) machine delivers oxygen into the nose or nose and mouth via a mask. Delivering pressurized oxygen into the mask keeps the airway open and aids in the absorption of oxygen.

Further, a bilevel positive airway pressure (BIPAP) machine pushes oxygen into the individual's lungs by keeping the lungs open and allowing more oxygen to enter. A BIPAP machine also uses a mask worn by the individual and supplies the needed pressure to keep the individual inhaling and exhaling so oxygen may enter the air sacs within the lungs.

A breathing machine like the CPAP and the BIPAP often treat obstructive sleep apnea. Obstructive sleep apnea is often caused by such diseases as congestive heart failure, high blood pressure and type 2 diabetes, polycystic ovary syndrome, hormonal disorders, a prior stroke, and chronic lung diseases, such as asthma.

Incorporated in the CPAP and the BIPBP are vent openings. The vent openings allow exhaled carbon dioxide (CO²) and superfluous oxygen to escape the mask. Unfortunately, the vent holes are not typically located in an ideal position. In this regard, the vent holes are usually positioned so that the exhaled CO² and superfluous oxygen are bothersome to the individual.

### BRIEF DESCRIPTION OF THE DRAWINGS

The present disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 is a front view of a prior art pressurized oxygen delivery tube and corresponding connecting elbow showing ventilation openings in the elbow for exhaled carbon dioxide (CO²) and superfluous oxygen.

FIG. 2 is a side view of the prior art pressurized oxygen delivery tube and the corresponding connecting elbow showing the ventilation openings in the elbow as shown in FIG. 1.

FIG. 3 is a back view of the prior art pressurized oxygen delivery tube and the corresponding connecting elbow showing the ventilation openings in the elbow as shown in FIG. 1.

FIG. 4 is a back view of a prior art oxygen delivery device using a mask for delivering pressurized oxygen to an individual, and the mask comprises vent holes for exhaled carbon dioxide (CO²) and superfluous oxygen.

FIG. 5 is a side view of the prior art oxygen delivery device for delivering pressurized oxygen to the individual as shown in FIG. 4.

FIG. 6 is a front view of an exemplary tube attachment in accordance with an embodiment of the present disclosure to be attached to an oxygen delivery system having vent holes at an unattached end of tubing to expel exhaled CO² and superfluous oxygen.

FIG. 7 is a side view of the tube attachment for the oxygen delivery system having vent holes at the unattached end of the tubing to expel exhaled CO² and superfluous oxygen as shown in FIG. 6.

FIG. 8 is a back view of the tube attachment for the oxygen delivery system having vent holes at the unattached end of the tubing to expel exhaled CO² and superfluous oxygen as shown in FIG. 6.

FIG. 9 is a front view of an exemplary mask attachment in accordance with an embodiment of the present disclosure to be attached to an oxygen delivery system having vent holes at an unattached end of tubing to expel exhaled CO² and superfluous oxygen.

FIG. 10 is a side view of the mask attachment for the oxygen delivery system having vent holes at the unattached end of the tubing to expel exhaled CO² and superfluous oxygen as shown in FIG. 9

FIG. 11 is a back view of the mask attachment for the oxygen delivery system having vent holes at the unattached end of the tubing to expel exhaled CO² and superfluous oxygen as shown in FIG. 9.

FIG. 12 is a side view of a device for delivering normalized oxygen to an individual and showing the tube attachment shown in FIG. 7 in accordance with an embodiment of the present disclosure attached to an oxygen delivery tube to divert CO² and superfluous oxygen from a plurality of vent holes in the connecting elbow to an unattached end of the tube attachment.

FIG. 13 is a side view of a device for delivering normalized oxygen to an individual and showing a tube attachment shown in FIG. 10 in accordance with an embodiment of the present disclosure attached to a mask to divert CO² and superfluous oxygen from a plurality of vent holes in the mask to an unattached end of the tube attachment.

### BRIEF DESCRIPTION

The present disclosure is a tube attachment that is configured to be attached to an oxygen delivery tube of a device for delivering normalized oxygen to an individual, e.g., a continuous positive airway pressure (C-PAP) machine or a bilevel positive airway pressure (BIPAP) machine. In one embodiment, an attachment end of the tube attachment attaches to an elbow of the device for delivering normalized oxygen and envelops vent holes formed in the elbow of the device for delivering normalized oxygen. A corrugated tube of the exemplary attachment diverts exhaled CO² and superfluous oxygen to a passive opposing end of the tube attachment and passes the CO² and superfluous oxygen through vent holes formed in the passive opposing end of the tube attachment.

In another embodiment, the prior art device for delivering normalized oxygen to the individual may have vent holes formed in a mask worn by the individual. In such a scenario, an exemplary attachment in accordance with an embodiment of the present disclosure envelops the vent holes formed in the mask. A corrugated tube attached to the elbow diverts exhaled CO² and superfluous oxygen to a passive opposing end of the tube attachment and passes the CO² and superfluous oxygen through vent holes formed in the passive opposing end of the tube attachment.

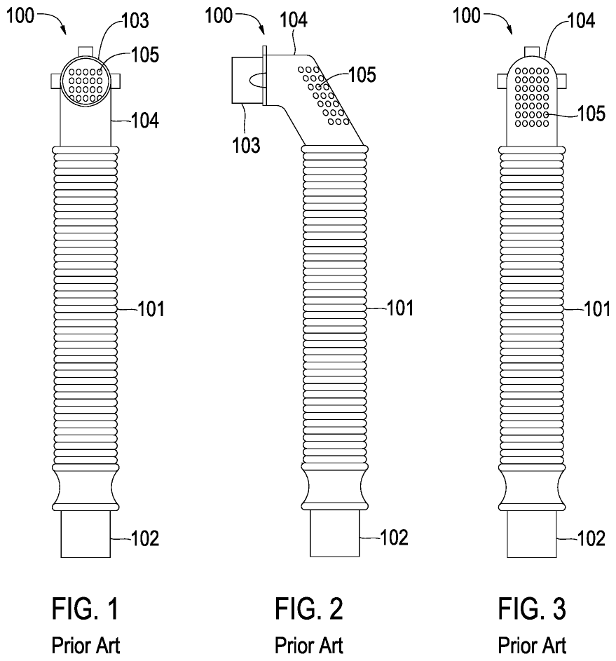

FIG. 1 is a front view of a prior art pressurized oxygen delivery tube 100 having a corresponding connecting elbow tube 104 showing ventilation openings 105 in the elbow tube 104 for a device for delivering pressurized oxygen to an individual. The elbow tube 104 couples to a mask (not shown) by a connector 103. The mask covers the individual's nose and delivers the oxygen to the individual. The prior art oxygen delivery tube 100 comprises the corrugated tube 101 that is coupled to the device (not shown) via a connector 102 for delivering pressurized oxygen to the individual.

Note that the device for delivering pressurized oxygen to the individual is any type of device that takes in oxygen from its environment, filters the oxygen, pressurizes it, and delivers it through a tube to a mask worn by an individual.

In operation, a connector 102 connects to the device for delivering pressurized oxygen to the individual. The device for delivering pressurized oxygen to the individual takes in oxygen from its environment, filters the oxygen, pressurizes it, and forces the oxygen through the corrugated tube 101. The oxygen flows through the corrugated tube through the elbow tube 104 and into the mask (not shown). The individual inhales the delivered oxygen then exhales CO² and superfluous oxygen. The vent holes 105 in the elbow tube 104 allows most of the CO² and the superfluous oxygen to escape from the tube 100.

FIG. 2 is a side view of the prior art pressurized oxygen delivery tube 100 having the corresponding connecting elbow tube 104 showing the ventilation openings 105 in the elbow tube 104. As indicated herein, the pressurized oxygen delivery tube 100 comprises a connector 103 that connects the pressurized oxygen delivery tube 100 to a mask (not shown) to deliver the pressurized oxygen to the individual. Additionally, the connector 102 couples the corrugated tube 101 to the device (not shown) via a connector 102 for delivering pressurized oxygen to the individual. Notably, the vent holes 105 are formed on a surface of the elbow tube 104. When the individual exhales, CO² and superfluous oxygen escape via the vent holes 105.

FIG. 3 is a back view of the prior art pressurized oxygen delivery tube 100 having the corresponding connecting elbow tube 104 showing the ventilation openings 105 in the elbow tube 104. As indicated herein, the pressurized oxygen delivery tube 100 comprises a connector (not shown from this perspective) that connects the pressurized oxygen delivery tube 100 to a mask (not shown) to deliver the pressurized oxygen to the individual. Additionally, the connector 102 couples the corrugated tube 101 to the device (not shown) via a connector 102 for delivering pressurized oxygen to the individual. Notably, the vent holes 105 are formed on a surface of the elbow tube 104. When the individual exhales CO² superfluous oxygen escape via the vent holes 105 so that the individual does not inhale the CO².

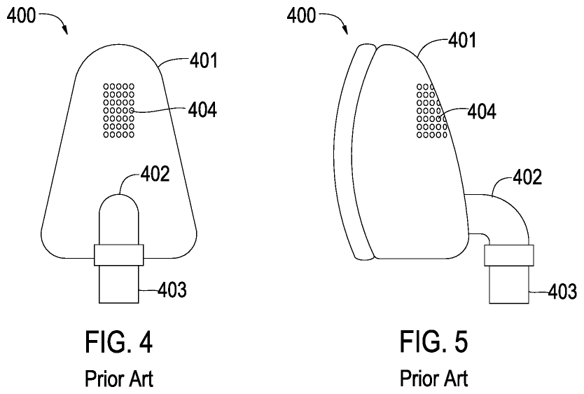

FIG. 4 is a back view of the prior art oxygen delivery device 400 using a mask 401 for delivering pressurized oxygen to an individual (not shown). The mask 401 comprises vent holes 404 for removing exhaled carbon dioxide (CO²) and superfluous oxygen so that the individual does not inhale the CO². The oxygen delivery device 400 comprises an elbow tube 402 that connects via a connector 403 to a corrugated tube (not shown) that connects to a device for delivering pressurized oxygen to the mask 401.

FIG. 5 is a side view of the prior art oxygen delivery device 400 using the mask 401 for delivering pressurized oxygen to an individual (not shown). The mask 401 comprises the vent holes 404 for removing exhaled carbon dioxide (CO²) and superfluous oxygen so that the individual does not inhale the CO². The oxygen delivery device 400 comprises an elbow tube 402 that connects via a connector 403 to a corrugated tube (not shown) that connects to a device for delivering pressurized oxygen to the mask 401.

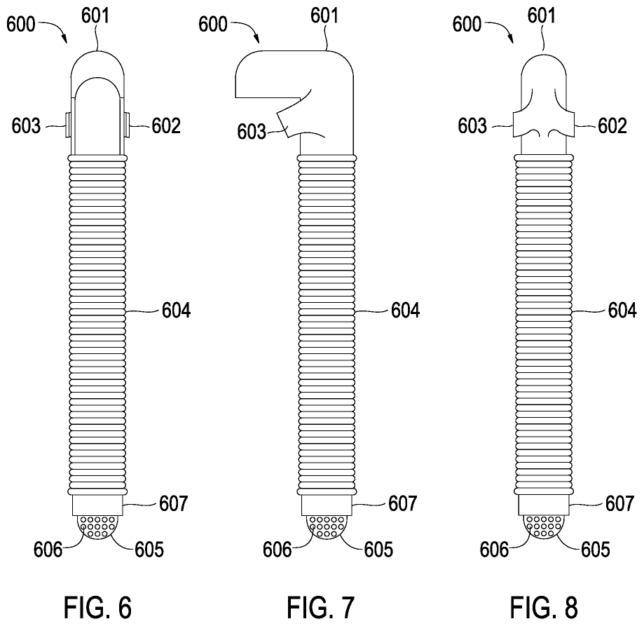

FIG. 6 is a front view of an exemplary tube attachment and CO2 and superfluous oxygen distribution unit 600 in accordance with an embodiment of the present disclosure to be attached to an oxygen delivery system (not shown). The tube attachment and CO2 and superfluous oxygen distribution unit 600 comprise one or more attachment flaps 602 and 603. The attachment flaps 602 and 603 attach to the pressurized oxygen delivery tube 100 (FIG. 1). In one embodiment, the tabs comprise a rubberized sealant, glue, or other airtight material to ensure that CO² and superfluous oxygen do not escape prior to being expelled.

The tube attachment and CO² and superfluous oxygen distribution unit 600 comprises an elbow 601 contiguous with the attachment flaps 602 and 603. The attachment flaps 602 and 603 wrap around the pressurized oxygen delivery tube 104 and the elbow tube covers vent holes 105 (FIG. 1) in the pressurized oxygen delivery tube 100.

Further, the tube attachment and CO2 and superfluous oxygen distribution unit 600 comprises a corrugated tube 604 that couples to the elbow tube 601. On an unattached end 607 of the corrugated tube 604 is a half sphere 605. In the half sphere 605 is a plurality of vent holes 606. Note that a half sphere 605 is shown, but it may be other shapes and sizes in other embodiments.

In operation, the individual exhales CO2 and superfluous oxygen. The CO² and superfluous oxygen transfer from the elbow tube 104 (FIG. 1) to the elbow tube 601. The CO²and the superfluous oxygen travel through the corrugated tube 604 and out the vent holes 606. Because the vent holes 606 are positioned away from an individual's face or head, the escaping CO² and superfluous oxygen do not tend to annoy or aggravate the individual using the system for delivering pressurized oxygen to the individual.

FIG. 7 is a side view of the exemplary tube attachment and CO2 and superfluous oxygen distribution unit 600 in accordance with an embodiment of the present disclosure to be attached to an oxygen delivery system (not shown). The tube attachment and CO2 and superfluous oxygen distribution unit 600 comprises the one or more attachment flaps 602 and 603. The attachment flaps 602 and 603 wrap around and attach to the pressurized oxygen delivery tube 100 (FIG. 2).

The tube attachment and CO2 and superfluous oxygen distribution unit 600 comprises the elbow 601 contiguous with the attachment flaps 602 and 603. The attachment flaps 602 and 603 wrap around the pressurized oxygen delivery tube 100 and the elbow tube covers vent holes 105 (FIG. 2) in the pressurized oxygen delivery tube 100.

Further, the tube attachment and CO2 and superfluous oxygen distribution unit 600 comprises a corrugated tube 604 that couples to the elbow tube 601. On an unattached end 607 of the corrugated tube 604 is a half sphere 605. Formed in the half sphere 605 is a plurality of vent holes 606.

In operation, the individual exhales CO² and superfluous oxygen. The CO² and superfluous oxygen transfer from the elbow tube 104 (FIG. 2) to the elbow tube 601. The CO²and the superfluous oxygen travel through the corrugated tube 604 and out the vent holes 606. Because the vent holes 606 are positioned away from an individual's face or head, the escaping CO² and superfluous oxygen do not tend to annoy or aggravate the individual using the system for delivering pressurized oxygen to the individual.

FIG. 8 is a back view of the exemplary tube attachment and CO2 and superfluous oxygen distribution unit 600 in accordance with an embodiment of the present disclosure to be attached to an oxygen delivery system (not shown). The tube attachment and CO2 and superfluous oxygen distribution unit 600 comprises the one or more attachment flaps 602 and 603. The attachment flaps 602 and 603 wrap around and attach to the pressurized oxygen delivery tube 100 (FIG. 3).

The tube attachment and CO2 and superfluous oxygen distribution unit 600 comprises the elbow 601 contiguous with the attachment flaps 602 and 603. The attachment flaps 602 and 603 wrap around the pressurized oxygen delivery tube 100 and the elbow tube covers vent holes 105 (FIG. 3) in the pressurized oxygen delivery tube 100.

Further, the tube attachment and CO2 and superfluous oxygen distribution unit 600 comprises a corrugated tube 604 that couples to the elbow tube 601. On an unattached end 607 of the corrugated tube 604 is a half sphere 605. Formed in the half sphere 605 is a plurality of vent holes 606.

In operation, the individual exhales CO² and superfluous oxygen. The CO² and superfluous oxygen transfer from the elbow tube 104 (FIG. 3) to the elbow tube 601. The CO²and the superfluous oxygen travel through the corrugated tube 604 and out the vent holes 606. Because the vent holes 606 are positioned away from an individual's face or head, the escaping CO² and superfluous oxygen do not tend to annoy or aggravate the individual using the system for delivering pressurized oxygen to the individual.

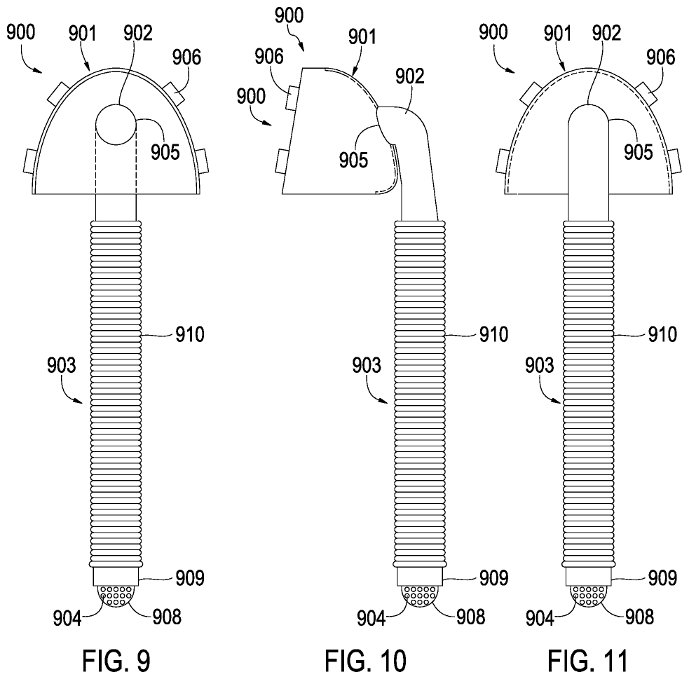

FIG. 9 is a front view of an oxygen delivery system 900 in accordance with another embodiment of the present disclosure showing a mask 901 attached to the tube attachment and CO2 and superfluous oxygen distribution unit 903 via the elbow tube 902 in accordance with an embodiment of the present disclosure. The mask 901 attaches over vent holes 404 (FIG. 4) in the mask 400 (FIG. 4) via connectors 906.

The tube attachment and CO2 and superfluous oxygen distribution unit 900 further comprises the corrugated tube 910. On the unattached end of the corrugated tube 910 is a half sphere 908. Formed in the half sphere 908 is the plurality of vent holes 904. The half sphere 908 is coupled to the corrugated tube 910 via a collar 909.

In this regard, the elbow tube 902 attaches to an opening 905 in the mask 901. The mask 901 covers the mask 401 thereby covering the vent holes 404 in the mask. The individual is delivered oxygen, then the individual exhales CO² and superfluous oxygen. The CO² and superfluous oxygen transfer from the mask 401 (FIG. 4) to the mask 901. The CO² and the superfluous oxygen travel through the corrugated tube 910 and out the vent holes 904. Because the vent holes 904 are positioned away from an individual's face or head, the escaping CO² and superfluous oxygen do not tend to annoy or aggravate the individual using the system for delivering pressurized oxygen to the individual.

FIG. 10 is a side view of the oxygen delivery system 900 in accordance with an embodiment of the present disclosure showing the mask 901 attached to the tube attachment and CO2 and superfluous oxygen distribution unit 903 via the elbow tube 902 in accordance with an embodiment of the present disclosure. The mask 901 attaches over vent holes 404 (FIG. 5) in the mask 400 (FIG. 5) via connectors 906.

The tube attachment and CO2 and superfluous oxygen distribution unit 900 further comprises the corrugated tube 910. On the unattached end of the corrugated tube 910 is a half sphere 908. Formed in the half sphere 908 is the plurality of vent holes 904. The half sphere 908 is coupled to the corrugated tube 910 via a collar 909.

In this regard, the elbow tube 902 attaches to an opening 905 in the mask 901. The mask 901 covers the mask 401 thereby covering the vent holes 404 in the mask. The individual is delivered oxygen, then the individual exhales CO² and superfluous oxygen. The CO² and superfluous oxygen transfer from the mask 401 (FIG. 5) to the mask 901. The CO² and the superfluous oxygen travel through the corrugated tube 910 and out the vent holes 904. Because the vent holes 904 are positioned away from an individual's face or head, the escaping CO² and superfluous oxygen do not tend to annoy or aggravate the individual using the system for delivering pressurized oxygen to the individual.

FIG. 11 is a back view of the oxygen delivery system 900 in accordance with an embodiment of the present disclosure showing the mask 901 attached to the tube attachment and CO2 and superfluous oxygen distribution unit 903 via the elbow tube 902 in accordance with an embodiment of the present disclosure. The mask 901 attaches over vent holes 404 (FIG. 4) in the mask 400 (FIG. 4) via connectors 906.

The tube attachment and CO2 and superfluous oxygen distribution unit 903 further comprises the corrugated tube 910. On the unattached end of the corrugated tube 910 is a half sphere 908. Formed in the half sphere 908 is the plurality of vent holes 904. The half sphere 908 is coupled to the corrugated tube via a collar 909.

In this regard, the elbow tube 902 attaches to an opening 905 in the mask 901. The mask 901 covers the mask 401 thereby covering the vent holes 404 in the mask. The individual is delivered oxygen, then the individual exhales CO² and superfluous oxygen. The CO² and superfluous oxygen transfer from the mask 401 (FIG. 4) to the mask 901. The CO² and the superfluous oxygen travel through the corrugated tube 910 and out the vent holes 904. Because the vent holes 904 are positioned away from an individual's face or head, the escaping CO² and superfluous oxygen do not tend to annoy or aggravate the individual using the system for delivering pressurized oxygen to the individual.

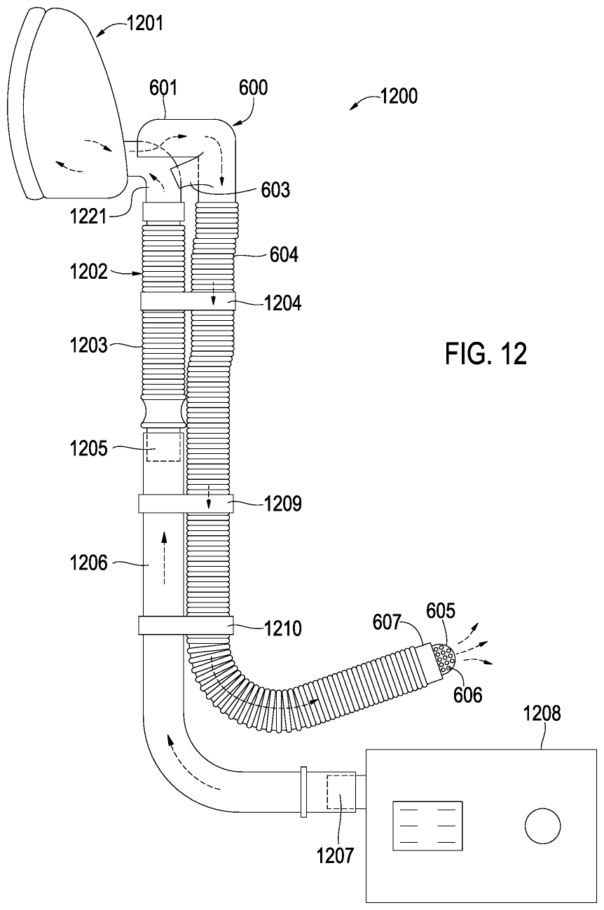

FIG. 12 is an exemplary pressurized oxygen delivery system 1200 in accordance with an embodiment of the present disclosure. The pressurized oxygen delivery system 1200 comprises an oxygen delivery system 1202 and an exhalation distribution system and CO² and superfluous oxygen distribution unit 600.

The pressurized oxygen delivery system 1200 comprises an oxygen pressurization unit 1208. As described above, the pressurization unit 1208 may be, for example, a continuous positive airway pressure (C-PAP) machine or a bilevel positive airway pressure (BIPAP). Notably, the pressurization unit 1208 is any type of device that takes in oxygen from its environment, filters the oxygen, pressurizes the oxygen, and delivers the oxygen to an individual.

The pressurization unit 1208 is coupled to an initial tube 1206 via a connector 1207. The initial tube may be made of a flexible or in flexible plastic; however, other materials and other characteristics may be used in other embodiments.

The initial tube 1206 is coupled to an oxygen delivery corrugated tube 1203 via a connector 1205. The oxygen delivery corrugated tube 1203 is coupled to an elbow tube 1221. The oxygen delivery corrugated tube 1203 delivers oxygen to a mask 1201. While not shown in FIG. 12, the oxygen delivery elbow tube 1221 comprises vent holes 105 (FIG. 2) through which CO² and superfluous oxygen are eliminated when the individual exhales.

The pressurized oxygen delivery system 1200 further comprises the CO2 and superfluous oxygen distribution unit 600. The elbow tube 601 of the CO2 and superfluous oxygen distribution unit 600 wraps around and is fixedly coupled to the oxygen delivery elbow tube 1221 encompassing the vent holes 105 (FIG. 2) in the oxygen delivery elbow tube 1221.

The CO2 and superfluous oxygen distribution unit 600 comprises the corrugated tube 604 through which CO² and superfluous oxygen flow when exhaled by the individual. The CO2 and superfluous oxygen travel through the corrugated tube to the half sphere 605 where it is expelled through the vent holes 606.

In one embodiment, the oxygen delivery system 1202 and the CO2 and superfluous oxygen distribution unit 600 may be coupled together for ease of use. In such an embodiment, one or more ties, bands, or other couplers may be used. In FIG. 12, the oxygen delivery system 1202 and an CO2 and superfluous oxygen distribution unit 600 are coupled together via bands 1204, 1209, and 1210, e.g., metal bands or flexible bands.

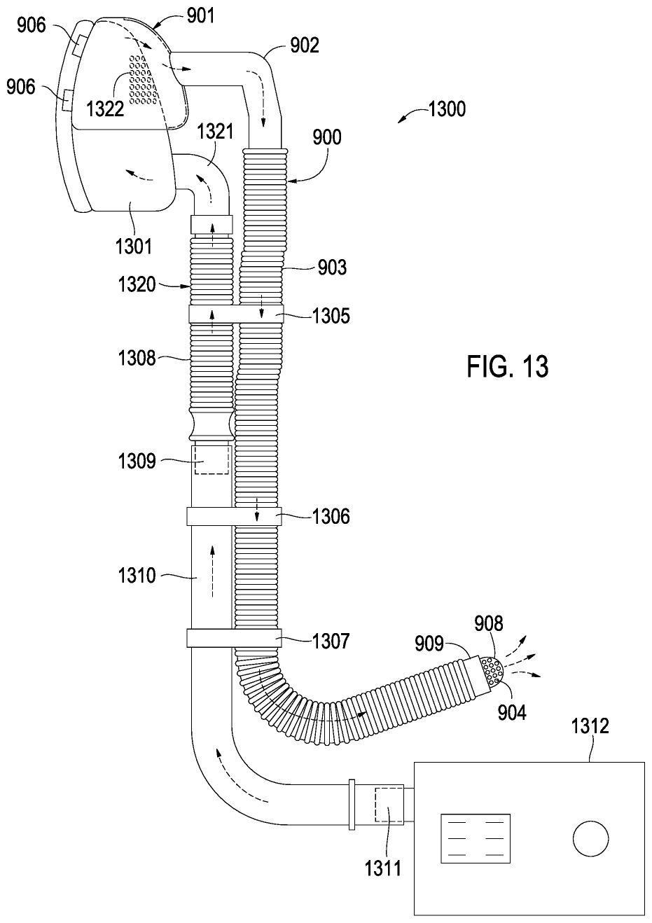

FIG. 13 is an exemplary pressurized oxygen delivery system 1300 in accordance with an embodiment of the present disclosure. The pressurized oxygen delivery system 1300 comprises an oxygen delivery system 1320 and an CO2 and superfluous oxygen distribution unit 900 as described in FIG. 9.

The pressurized oxygen delivery system 1320 comprises an oxygen pressurization unit 1312. As described above, the pressurization unit 1312 may be, for example, a continuous positive airway pressure (C-PAP) machine or a bilevel positive airway pressure (BIPAP). Notably, the pressurization unit 1312 is any type of device that takes in oxygen from its environment, filters the oxygen, pressurizes the oxygen, and delivers the oxygen to an individual. The pressurization unit 1312 is coupled to an initial tube 1310 via a connector

1311. The initial tube 1310 may be made of a flexible or in flexible plastic; however, other materials and other characteristics may be used in other embodiments.

The initial tube 1310 is coupled to an oxygen delivery corrugated tube 1308 via a connector 1309. The oxygen delivery corrugated tube 1308 is coupled to an elbow tube 1321. The oxygen delivery corrugated tube 1308 delivers oxygen to a mask 1301. The mask 1301 comprises a plurality of vent holes 1322 through which CO² and superfluous oxygen exit when the individual exhales.

The pressurized oxygen delivery system 1300 further comprises the CO2 and superfluous oxygen distribution unit 900. The elbow tube 902 of the CO2 and superfluous oxygen distribution unit 900 attaches to the secondary mask 901. The secondary mask 901 attaches to the mask 1301 via a plurality of connectors 906. The secondary mask 901 encompasses and covers the plurality of vent holes 1322.

The CO² and superfluous oxygen distribution unit 900 comprises the corrugated tube 903 through which CO² and superfluous oxygen flow when exhaled by the individual. The CO² and superfluous oxygen travel through the corrugated tube 903 to the half sphere 908 where it is expelled through vent holes 904.

In one embodiment, the oxygen delivery system 1320 and the CO2 and superfluous oxygen distribution unit 900 may be coupled together for ease of use. In such an embodiment, one or more ties, bands, or other couplers may be used. In FIG. 13, the oxygen delivery system 1320 and an CO2 and superfluous oxygen distribution unit 900 are coupled together via bands 1305, 1306, and 1307, e.g., metal bands or flexible bands.

What I claim is:

1. A device, comprising:
an attachment section configured for attaching to a pressurized oxygen delivery system and covering vent holes on the pressurized oxygen delivery system configured for venting exhaled carbon dioxide (CO²);
a tube fixedly coupled to the attachment section on a first end of the tube; and
a plurality of vent holes on a second end of the tube configured for eliminating the CO².

2. The device of claim 1, wherein the attachment section is an elbow tube having attachment tabs for wrapping around an elbow tube of the pressurized oxygen delivery system and covering the vent holes on the pressurized oxygen delivery system.

3. The device of claim 1, wherein the attachment section is a mask having attachment tabs for attaching to a mask of the pressurized oxygen delivery system and covering the vent holes on the pressurized oxygen delivery system.

4. The device of claim 1, wherein the attachment section comprises a plurality of tabs for wrapping around an elbow tube of the pressurized oxygen delivery system.

5. The device of claim 1, wherein the attachment section comprises a plurality of tabs for wrapping around a mask of the pressurized oxygen delivery system.

6. The device of claim 1, wherein the attachment section comprises a plurality of tabs for wrapping around an elbow tube of the pressurized oxygen delivery system and the tabs comprise an airtight structure configured for not allowing CO² to escape before being expelled.

7. The device of claim 1, wherein the attachment section comprises a plurality of tabs for connecting to a mask of the pressurized oxygen delivery system and the tabs comprise an airtight structure configured for not allowing CO² to escape before being expelled.

8. The device of claim 1, wherein the vent holes on the second end of the tube are formed in a half sphere.

9. The device of claim 1, further comprising a plurality of connectors for attaching to an oxygen delivery system.

10. The device of claim 9, wherein the connectors are metal bands.

11. The device of claim 9, wherein the connectors are flexible.

12. A system for delivering pressurized oxygen to an individual, comprising:
an oxygen pressurization unit;
an oxygen delivery tube coupled to the oxygen pressurization unit;
a mask coupled to the oxygen delivery tube for delivering the oxygen to the individual;
a plurality of vent holes;
an attachment section covering the plurality of vent holes configured for capturing CO2;
an exhalation tube having a first end coupled to the attachment section and a second end; and
a plurality of vent holes on the second end of the tube configured for eliminating CO2 away from the individual's face.

13. The system of claim 12, wherein the oxygen delivery tube is an elbow tube for delivering oxygen and the attachment section has an elbow tube having attachment tabs for wrapping around the elbow tube for delivering oxygen and covering the vent holes on the elbow tube for delivering oxygen.

14. The system of claim 12, wherein the attachment section is a mask having attachment tabs for attaching to the mask coupled to the oxygen delivery tube and covering the vent holes on the mask coupled to the oxygen delivery tube.

15. The system of claim 12, wherein the attachment section comprises a plurality of tabs for wrapping around the oxygen delivery tube.

16. The system of claim 12, wherein the attachment section comprises a plurality of tabs for wrapping around the mask coupled to the oxygen delivery tube.

17. The device of claim 12, wherein the attachment section comprises a plurality of tabs for wrapping around oxygen delivery tube and the tabs comprise an airtight structure configured for not allowing CO² to escape before being expelled.

18. The system of claim 12, wherein the attachment section comprises a plurality of tabs for connecting to a mask coupled to the oxygen delivery tube and the tabs comprise an airtight structure configured for not allowing CO² to escape before being expelled.

19. The system of claim 12, wherein the vent holes on the second end of the tube are formed in a half sphere.

20. The system of claim 12, wherein the oxygen delivery tube is coupled to the exhalation tube.
